(19) United States
(12) Patent Application Publication
(10) Pub. No.: US 2025/0127832 A1
(43) Pub. Date: Apr. 24, 2025

(54) Lactobacillus Plantarum and Application Thereof in Production of Urolithin A

(71) Applicant: Jiangnan University, Wuxi (CN)

(72) Inventors: Shumao CUI, Wuxi (CN); Xin TANG, Wuxi (CN); Mengwei ZHANG, Wuxi (CN); Bingyong MAO, Wuxi (CN); Qiuxiang Zhang, Wuxi (CN); Bo YANG, Wuxi (CN); Jianxin ZHAO, Wuxi (CN); Wei CHEN, Wuxi (CN)

(21) Appl. No.: 18/989,721

(22) Filed: Dec. 20, 2024

Related U.S. Application Data
(63) Continuation of application No. PCT/CN2023/127174, filed on Oct. 27, 2023.

(30) Foreign Application Priority Data
Nov. 10, 2022 (CN) 2022114097204

(51) Int. Cl. A61K 35/747; A61P 43/00; C12N 1/20; C12P 17/06; C12R 1/25

(52) U.S. Cl. CPC A61K 35/747; A61P 43/00; C12N 1/205; C12P 17/06; C12R 2001/25

(57) ABSTRACT

Disclosed is a Lactobacillus plantarum and an application thereof in the production of urolithin A, belonging to the technical field of biology. The disclosure provides a Lactiplantibacillus plantarum CCFM1290 preserved in Guangdong Microbial Culture Collection Center (GDMCC) with a preservation number of GDMCC No: 62801 and a preservation date of Sep. 15, 2022. The L. plantarum CCFM1290 may convert ETs into various Uros represented by Uro-A. A fermented product or compound preparation provided by the disclosure may relieve muscle function damage. Compared with a control group, the fermented product and compound preparation may improve the swimming capability of Caenorhabditis elegans by 33.84% and 27.99%. Finally, Uro-type substances such as Uro-A obtained by this biosynthesis method may be applied to functional food development to achieve beneficial effects of delaying senescence, keeping muscles healthy, etc.

### TECHNICAL FIELD

The disclosure herein relates a Lactobacillus plantarum and its application thereof in the production of urolithin A, belonging to the technical field of biology.

### BACKGROUND

Ellagitannins (ETs) is a polar macromolecular substance and is difficult to be directly absorbed by the gastrointestinal tract, so its bioavailability is very low. ETs is hydrolyzed into ellagic acid (EA) in the small intestine, then, EA directly reaches the distal end (colon) of the gastrointestinal tract and is metabolized by colonizing intestinal flora to become more easily absorbed dibenzopyrane-6-one derivatives, Urolithin (Uro). Uro enters the bloodstream in the form of phase II conjugates (glucuronides and sulfates) under the effect of human cell enzymes, reaches tissues throughout the body within 3 to 4 days after intake to trigger biological effects, and is finally bound in the livers and excreted in urine. Uro is a true bioactive substance in ETs-rich foods taking their effects in living organisms (disclosed in Journal of Agricultural and Food Chemistry, 2005, 53 (02): 227-235).

Intestinal floras of different persons have specificity, so produced metabolites have significant differences between individuals. According to a study by Tomás-Barberán et al in 2014, based on different urolithin excretion abilities of human subjects, urolithin metabotypes (UMs) may be divided into three phenotypes: “phenotype A”, “phenotype B”, and “phenotype 0”. The metabolic phenotype A (UM-A) produces Uro-A, the metabolic phenotype B (UM-B) mainly produces iso-Uro-A and Uro-B, and also produces a small amount of Uro-A, and the metabolic phenotype 0 (UM-0) produces no Uro-A, no iso-Uro-A and no Uro-B (disclosed in Journal of Agricultural and Food Chemistry, 2014, 62 (28): 6535-8). In recent years, a new study has shown that UMs has a certain correlation with the age. For young healthy people (5 to 30 years old), UM-A accounts for about 70% to 80%, and UM-B accounts for about 10% to 20%. With the age increasing (30 to 90 years old), UM-A decreases and UM-B increases, and their proportions finally reach 50% and 40%. However, the proportion (10%) of UM-O remains constant from the age of 5 years old to the age of 90 years old (disclosed in Food & Function, 2018, 9 (8): 4100-4106). It further indicates that there are significant differences about Uro in human body metabolism.

Various studies have shown that Uro-A in Uro has important effects in aspects of anti-aging, anti-oxidation, anti-inflammation, anti-cancer, obesity prevention, estrogen receptor regulation, etc. Especially in recent years, the studies of Uro-A in an anti-aging aspect have received much attention. A nematode study in Nature Medicine in 2016 showed that Uro-A may remove damaged mitochondria and regulate mitochondrial biosynthesis to thus prolong the lifespan of Caenorhabditis elegans and achieve the anti-aging effect (disclosed in Nature Medicine, 2016, 22 (8): 879-888). A human clinical trial in Nature Metabolism in 2019 demonstrated the anti-aging efficacy and safety of Uro-A directly taken orally by humans (disclosed in Nature Metabolism, 2019, 1 (6): 595-603). Recently, researchers have begun to research the effect of Uro-A on relieving mitochondria-related diseases. A Duchenne muscular dystrophy model mouse trial in Science Translational Medicine in 2021 demonstrated that Uro-A might improve the muscle function and prolong the lifespan (disclosed in Science Translational Medicine, 2021, 13 (588): 12).

The United States Food and Drug Administration has certified Uro-A as GRAS (Generally Recognized as Safe). At present, the only industrial production method of Uro-A is chemical synthesis. According to chemical synthesis, 3-methoxybenzoic acid is used as a starting material to achieve batch preparation of Uro-A through chemical reactions such as a bromination reaction, a copper-catalyzed coupling esterification reaction, and a demethylation reaction (recorded in a Chinese invention patent application with the public number of CN 105142632A). However, Uro-A prepared by a chemical synthesis method cannot be applied to the addition in foods in China. A biological conversion method is low in cost, easy in operation, and can achieve the purpose of using Uro-A as a functional food raw material. At present, there has been only one document reported a strain capable of converting EA to produce Uro-A (yield: 0.42 μM (˜0.09576 mg/L), conversion rate: 3.5%): Bifidobacterium pseudocatenulatum INIA P815 (disclosed in Journal of Functional Foods, 2018, 45 (18): 95-99). Since EA is insoluble in water, it needs to be dissolved into dimethyl sulfoxide (DMSO) at first and then added to a culture medium. DMSO may inhibit the growth of bacteria, reduce enzyme activity, and finally influence the conversion of EA to Uro-A. Additionally, due to the existence of an organic solvent DMSO, it is difficult to be directly applied to the addition in functional foods.

### SUMMARY

The disclosure provides a probiotic being capable of converting ETs to produce Uro-A and being in a list of edible fungi and a method of producing urolithin A from the probiotic and studies a senescence delaying effect of a prepared fermented product and compound preparation to thus lay a foundation for industrialized production of senescence delaying products. According to the disclosure, the target probiotic has been widely and deeply studied, and a Lactobacillus plantarum capable of converting ETs to product Uro-A is finally discovered.

The disclosure provides a Lactiplantibacillus plantarum CCFM1290 preserved in Guangdong Microbial Culture Collection Center (GDMCC) with a preservation number of GDMCC No: 62801 and a preservation date of Sep. 15, 2022.

The L. plantarum CCFM1290 is derived from the feces of healthy people. Through sequencing analysis on the strain, a 16S rDNA sequence was as shown by SEQ ID NO.1. Through nucleic acid sequence alignment in NCBI on the obtained sequence, the result showed that the strain is L. plantarum of Lactobacillus, and is named as L. plantarum CCFM1290.

A bacterial colony of the L. plantarum CCFM1290 on a BHI solid medium is raised, white, smooth, and round, with a diameter of approximately 3 mm.

The disclosure provides a biosynthesis method of a urolithin substance. A strain capable of converting a fermentation substrate to produce a fermented product Uro (the strain is capable of converting ETs to produce Uro) is inoculated into a reaction system using a substance containing ellagitannins (ETs) or ellagic acid (EA) as a substrate to obtain a fermentation product rich of Uro, and then, the fermentation product rich of Uro is further subjected to separation purification to obtain a Uro extract. The urolithin substances are Urolithin A (dihydroxyl urolithin), Urolithin M5 (pentacarbonyl urolithin), Urolithin D (tetracarbonyl urolithin), Urolithin C (trihydroxyl urolithin), etc. or combination thereof.

In an embodiment of the disclosure, the strain capable of converting a fermentation substrate to produce a fermented product, Uro, is L. plantarum CCFM1290.

In an embodiment of the disclosure, according to the method, a substance containing ETs or EA is used as a substrate, and reaction is performed by using the L. plantarum CCFM1290 to obtain a reaction solution. Then, the urolithin A is extracted from the reaction solution. The L. plantarum CCFM1290 is preserved in Guangdong Microbial Culture Collection Center (GDMCC) with a preservation number of GDMCC No: 62801 and a preservation date of Sep. 15, 2022.

In an embodiment of the disclosure, the biosynthesis method involves using a Uro precursor substance, which may include, but is not limited to, one or more dietary sources such as ETs or EA.

In an embodiment of the disclosure, the substance containing ETs or EA includes, but is not limited to, one or a combination of more of the following: berries such as pomegranates, mulberries, raspberries, and strawberries, as well as nuts such as walnuts, pistachios and corcassian walnuts.

In an embodiment of the disclosure, the L. plantarum CCFM1290 is fermented under an aerobic condition.

In an embodiment of the disclosure, the L. plantarum CCFM1290 is fermented under a 150-200 rpm oscillation culture condition.

In an embodiment of the disclosure, the fermentation temperature is 30° C. to 37° C.

In an embodiment of the disclosure, the fermentation time is 24 h to 72 h.

In an embodiment of the disclosure, the concentration of ETs is higher than or equal to 1.5 g/L.

In an embodiment of the disclosure, the substrate is a BHI broth or pomegranate juice. In a case that the substrate is the BHI broth, additional ETs need to be added. In a case that the substrate is the NFC pomegranate juice, the pH value needs to be adjusted to 6.8 to 7.2 at first, and then, proper ingredients are added to maintain the pH value in a fermentation process and to provide a nitrogen source (10 g/L of tryptone, 5 g/L of yeast powder, 2 g/L of dipotassium phosphate and 2 g/L of ammonium citrate dibasic) required for the strain growth.

In an embodiment of the disclosure, a method of extracting a Uro ingredient from the fermentation broth mainly includes: firstly taking a certain amount of fermentation broth, respectively adding an equal amount of diethyl ether and ethyl acetate, continuously performing extraction 3 times, performing freeze vacuum concentration by centrifuging on an extraction solution till the extraction solution is dry, then, performing redissolution by using methyl alcohol (performing ultrasonic solubilization), finally, performing filtration through a 0.22 μm cellulose acetate filter, and performing detection through HPLC and LC-MS/MS.

The disclosure further provides an application of the L. plantarum or the method in the production of Uro-A. The application has the main performance of improving muscles and delaying senescence, and it may be applied in food, medicine or health care products.

The disclosure provides a method for preparing urolithin A. The method includes: using the L. plantarum CCFM1290, using a substance containing ETs or EA as a substrate, preparing a reaction solution through taking a reaction, and performing extraction from the reaction solution to obtain the urolithin A

In an embodiment of the disclosure, the substance containing the ETs includes: pomegranates, mulberry mulberries, raspberries, strawberries, walnuts, pistachio nuts or corcassian walnuts.

In an embodiment of the disclosure, the reaction conditions include a temperature of 30° C. to 37° C. and a revolution velocity of 150 rpm to 200 rpm.

In an embodiment of the disclosure, the reaction time is 24 h to 72 h.

In an embodiment of the disclosure, the concentration of ETs in a reaction substrate is higher than or equal to 1.5 g/L.

In an embodiment of the disclosure, the reaction substrate is NFC pomegranate juice.

In an embodiment of the disclosure, the pH value of the pomegranate juice is adjusted to 6.8 to 7.2, and then, proper ingredients are added to keep the pH value in the fermentation process and to provide a nitrogen source required for strain growth.

In an embodiment of the disclosure, the nitrogen source required for maintaining the pH value in the fermentation process and required for strain growth includes: 5 to 15 g/L of tryptone, 0 to 5 g/L of yeast powder, 1 to 3 g/L of dipotassium phosphate and 1 to 3 g/L of ammonium citrate dibasic.

In an embodiment of the disclosure, the nitrogen source includes: 10 g/L of tryptone, 5 g/L of yeast powder, 2 g/L of dipotassium phosphate, and 2 g/L of ammonium citrate dibasic.

In an embodiment of the disclosure, a method of extracting the urolithin A mainly includes: firstly taking a certain amount of fermentation broth, respectively adding an equal amount of diethyl ether and ethyl acetate, continuously performing extraction for 3 times, performing freeze vacuum concentration by centrifuging on an extraction solution till the extraction solution is dry, then, performing redissolution by using methyl alcohol (performing ultrasonic solubilization), finally, performing filtration through a 0.22 μm cellulose acetate filter, and performing detection through HPLC and LC-MS/MS.

The disclosure further provides a composition including the L. plantarum CCFM1290.

In an embodiment of the disclosure, the composition includes but is not limited to a food, a health care product, a medicine, a dietary supplement, or a microbial preparation.

In an embodiment of the disclosure, the microbial agent includes the L. plantarum CCFM1290, or includes a fermentation broth containing the L. plantarum CCFM1290, or freeze-dried powder containing the L. plantarum CCFM1290, or inactivated thalli containing the L. plantarum CCFM1290, or a lysate containing the L. plantarum CCFM1290, or an extract containing the L. plantarum CCFM1290.

In an embodiment of the disclosure, the content of the L. plantarum CCFM1290 in the microbial agent is not lower than 10⁹ CFU/mL or 10¹⁰ CFU/g.

In an embodiment of the disclosure, the food is health care food, or the food is a dairy product or a bean product or a fruit and vegetable product produced by using a starter culture containing the L. plantarum CCFM1290, or the food is a beverage or snacks containing the L. plantarum CCFM1290.

In an embodiment of the disclosure, the composition is a medicine capable of delaying senescence, and the medicine includes the L. plantarum CCFM1290 or the microbial agent.

In an embodiment of the disclosure, during the preparation of senescence-delaying fermented product rich in Uro from the L. plantarum CCFM1290, an effective amount of Uro or bacteria powder or pharmaceutically acceptable carriers may be prepared in various shapes, such as tablets, drinks, injections, and capsules according to practical requirements.

In an embodiment of the disclosure, the composition is a dietary supplement, and has main ingredients of the L. plantarum CCFM1290 and a diet rich in ETs, and the composition is a compound preparation.

In an embodiment of the disclosure, the compound preparation is obtained by mixing the L. plantarum CCFM1290 bacteria powder, pomegranate freeze-dried powder, and mulberry freeze-dried powder according to a mass ratio of 5:1.5:0.5, and may be used for dietary supplementation to promote ETs metabolism, Uro absorption, etc., so that the senescence delaying efficiency is achieved. The disclosure further provides an application of a composition prepared from one or combination of more of the L. plantarum CCFM1290, ETs, and Uro for preparing food aimed at senescence intervention or muscle function improvement.

The disclosure further provides an application or the L. plantarum CCFM1290 or the microbial agent in the preparation of senescence-delaying products.

In an embodiment of the disclosure, the product is a medicine or a health care product.

In an embodiment of the disclosure, the content of the L. plantarum CCFM1290 in the product is not lower than 10⁹ CFU/mL or 10¹⁰ CFU/g.

### Beneficial Effects

The disclosure provides a strain capable of producing Uro-A: L. plantarum CCFM1290. This strain has the following advantages (the following values are all mean values of values obtained through four parallel experiments):

(1) The strain is inoculated into a culture medium containing 1.5 g/L of ETs at an inoculation amount of 2% to be fermented for 48 h, so that the ETs may be converted into various Uros represented by Uro-A, the content of the Uro-A in the culture medium is 24.70±0.82 μM, and a conversion rate of the Uro-A is 8.59%±0.62%.

The fermented product and compound preparation developed based on the L. plantarum CCFM1290 according to the disclosure may achieve a senescence delaying effect, and the lifespan of Caenorhabditis elegans may be respectively prolonged by 32.05% and 21.88%.

(3) The fermented product or compound preparation provided by the disclosure may further relieve muscle function damage. Compared with a control group, the fermented product and compound preparation may improve the swimming capability of Caenorhabditis elegans by 33.84% and 27.99%. Finally, Uro-type substances such as Uro-A obtained by this biosynthesis method may be applied to functional food development to achieve beneficial effects of delaying senescence, keeping muscles healthy, etc.

(4) The L. plantarum CCFM1290 provided by the disclosure may inhibit the accumulation of Caenorhabditis elegans lipid droplets along with the age increase to achieve a lipid-decreasing effect.

### Biological Material Collection

A L. plantarum CCFM1290 has a taxonomic name of L. plantarum, and has been preserved in Guangdong Microbial Culture Collection Center (GDMCC) on Sep. 15, 2022 with a preservation number of GDMCC No: 62801, and a preservation address is 5F, Building 59, Yard 100, Xianlie Middle Road, Guangzhou, Guangdong Institute of Scientific Microbiology.

### BRIEF DESCRIPTION OF FIGURES

FIG. 1A shows a screening flow process of a target strain;

FIG. 1B shows metabolic flowchart of Uro-A;

FIG. 2 shows the colony characteristics of the target strain;

FIG. 3 shows an HPLC spectrum of a fermented product;

FIG. 4A shows a total ions current diagram of a fermented product;

FIG. 4B shows a mass chromatograph of Uro-A;

FIG. 4C shows a secondary mass spectrum of Uro-A;

FIG. 5 shows the change of an accumulation amount of Uro-A in fermented products in different growth periods;

FIG. 6 shows the influence of the fermented products or compound preparations on the lifespan of Caenorhabditis elegans;

FIG. 7 shows the influence of the fermented products or compound preparations on the motion ability of Caenorhabditis elegans;

FIG. 8 shows the influence of the L. plantarum CCFM1290 on the accumulation of Caenorhabditis elegans lipid droplets, (a) shows the accumulation condition of lipid droplets on the eighth day after Caenorhabditis elegans are fed with Escherichia coli and L. plantarum, and (b) shows the average optical density of the lipid droplets calculated by using ImageJ software; and

FIG. 9A shows a condition that the L. plantarum is labeled with FITC, and it can be known that the L. plantarum is successfully labeled through observation by an inverted fluorescent microscope;

FIG. 9B shows the in vivo fluorescence condition of Caenorhabditis elegans after the Caenorhabditis elegans are fed with FITC-labeled L. plantarum;

FIG. 9C shows the in vivo fluorescence condition of Caenorhabditis elegans after the Caenorhabditis elegans is fed with E. coli OP50.

### DETAILED DESCRIPTION

An animal model of the disclosure is Caenorhabditis elegans, the Caenorhabditis elegans is an ideal model for studying the occurrence and therapeutic mechanism of degenerative diseases. Through life experiments on the Caenorhabditis elegans, it can be found that fermented extracts of a strain capable of converting ellagitannins (ETs) to produce urolithin A may intervene the senescence of Caenorhabditis elegans, and a powerful study model is provided for further development and utilization in the field of senescence-delaying foods.

The disclosure will be further illustrated hereafter in combination with specific examples and drawings.

ETs (Punicalagin) used in the following examples was purchased from Sigma, and had the purity being higher than or equal to 98%. Uro-A in the following examples was purchased from Sigma, and had the purity being higher than or equal to 97%. NFC pomegranate juice, pomegranate freeze-dried powder, and mulberry freeze-dried powder in the following examples were purchased from Wuxi Auchan supermarket. Caenorhabditis elegans in the following examples was N2 wild-type Caenorhabditis elegans, and was derived from the Culture Collection Center of Jiangnan University Food Biotechnology Center.

Oil red O staining solutions in the following examples were purchased from Solarbio (Product number: G1262), dexamethasone was purchased from Macklin (Product number: D829854), fluorescein isothiocyanate (FITC) was purchased from Solarbio (Product number: F8070).

Culture Media in the Following Examples were as Follows:

BHI solid medium: 10.0 g/L of peptone, 12.5 g/L of dehydrated calf brain extract powder, 5.0 g/L of dehydrated beef heart extract powder, 5.0 g/L of sodium chloride, 2.0 g/L of glucose, 2.5 g/L of disodium hydrogen phosphate and 15.0 g/L of agar. The pH value was adjusted to 7.0±0.2 (25° C.).

BHI liquid medium: 10.0 g/L of peptone, 12.5 g/L of dehydrated calf brain extract powder, 5.0 g/L of dehydrated beef heart extract powder, 5.0 g/L of sodium chloride, 2.0 g/L of glucose and 2.5 g/L of disodium hydrogen phosphate. The pH value was adjusted to 7.0±0.2 (25° C.).

Pomegranate juice fermentation broth: NFC pomegranate juice, 10 g/L of tryptone, 5 g/L of yeast powder, 2 g/L of K₂HPO₄ and 2 g/L of ammonium citrate dibasic. The pH value was adjusted to 7.0±0.2 (25° C.).

NGM culture medium: 2.5 g/L of peptone, 3.0 g/L of sodium chloride, 0.111 g/L of calcium chloride, 0.12 g/L of magnesium sulfate, 0.005 g/L of cholesterol, 3.4 g/L of potassium dihydrogen phosphate and 17.0 g/L of agar. The pH value was adjusted to 7.3±0.2 (25° C.).

LB solid medium: 10.0 g/L of tryptone, 5.0 g/L of yeast extracts, 10.0 g/L of sodium chloride and 15.0 g/L of agar. The pH value was adjusted to 7.0±0.2 (25° C.).

LB liquid medium: 10.0 g/L of tryptone, 5.0 g/L of yeast extracts and 10.0 g/L of sodium chloride. The pH value was adjusted to 7.0±0.2 (25° C.).

Detection methods in the following examples were as follows:

### Uro-A Content Detection: The Content of Uro-A was Detected Through HPLC, and the Existence of a Product Uro-A was Further Confirmed by a Q Exactive LC-MS Instrument:

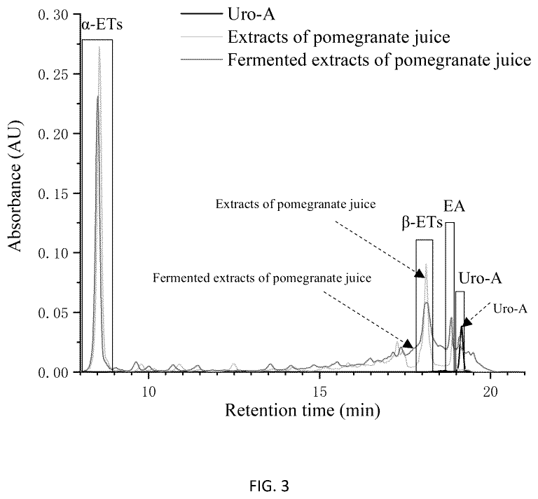

HPLC detection: A Waters 1525 liquid chromatograph and a liquid column X BridgeoRC18 (250×4.6 mm, 5 μm) were used. Mobile phase: 0.1% formic acid solution (phase A), and methyl alcohol (phase B). Spectrum scanning was performed to determine the maximum absorption wavelength. Detector: ultraviolet detector (UA) 306 nm. Elution condition: flow velocity of 1.0 ml/min, gradient elution. ETs had two structural isomers respectively being of an a type and a β type, and the two types of structural isomers might be continuously and mutually transformed. Appearance time of α-ETs: 8.5 min. Appearance time of β-ETs: 18.1 min. Appearance time of Uro-A: 19.1 min (FIG. 3).

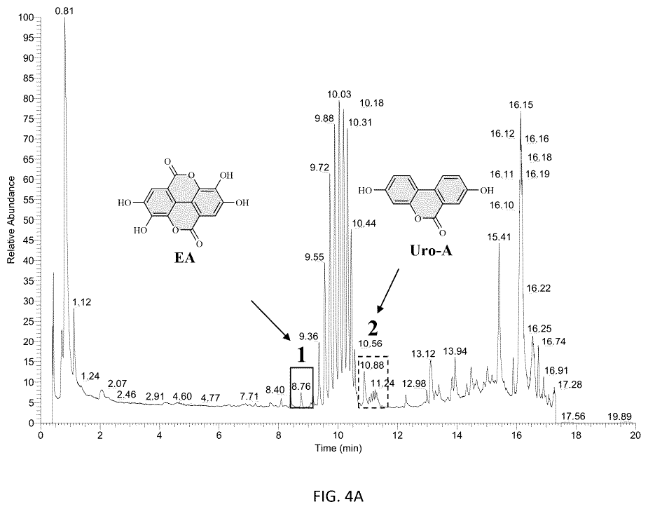

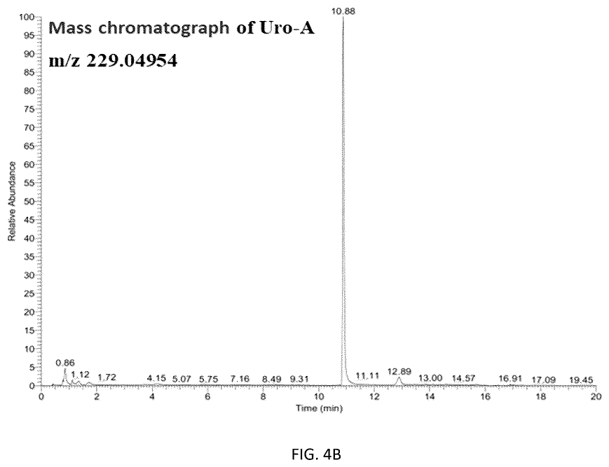

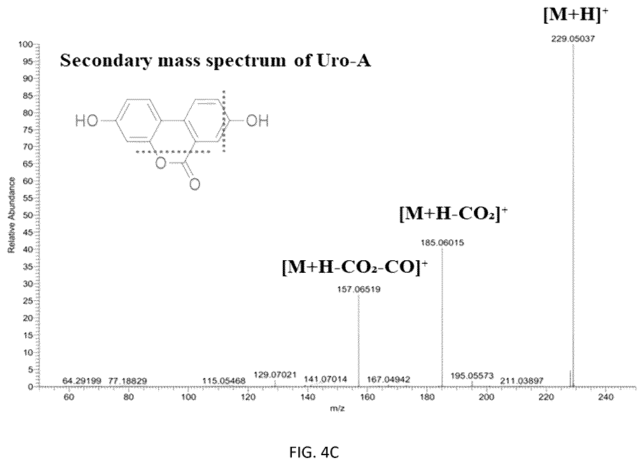

LCMS detection: a Q Exactive LC-MS instrument and a chromatographic column C18 were used. Mobile phase: 0.1% formic acid solution (phase A), and acetonitrile (phase B). EA, appearance time: 8.68 min, molecular formula: C₁₄H₆O₈, and actual m/z: 303.01184. Uro-A, appearance time: 10.88 min, molecular formula: C₁₃H₈O₄, and actual m/z: 229.04840. m/z of Uro-A secondary fragments: 229.05040, 185.06015 and 157.06517. (Relevant data of LCMS/MS is shown in FIG. 4).

### Example 1: Target Screening, Identification, Culture, Observation, and Preservation

### 1. Screening

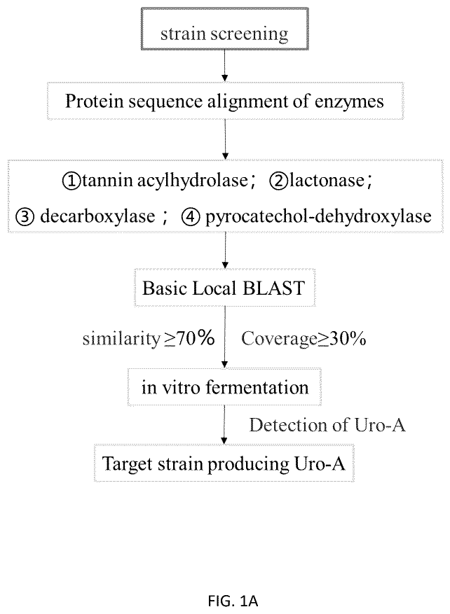

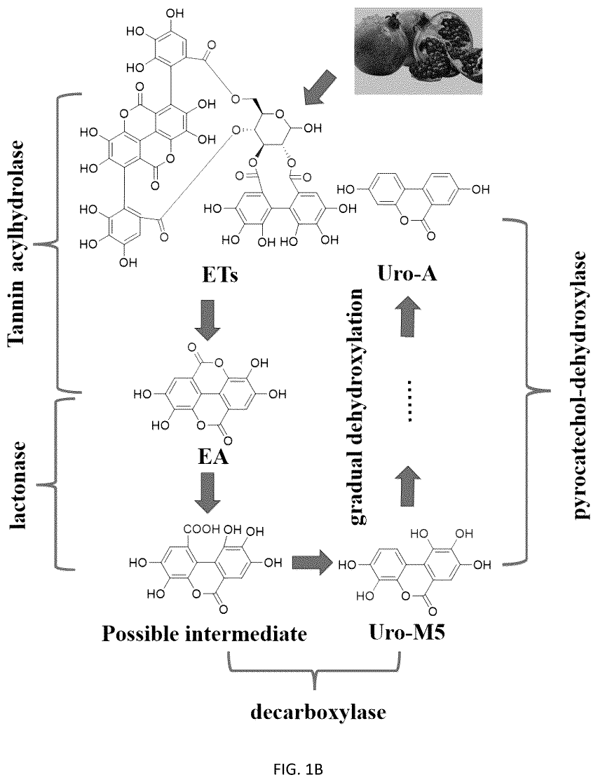

Tannin acylhydrolase (GenBank: AIR09580.1) may hydrolyze ester bonds and depside bonds in gallotannin to produce gallic acid and glucose, EA production through ETs hydrolysis is also achieved through such a path, that is, this enzyme may achieve an effect of hydrolyzing ETs to produce Uro-A. Additionally, a process of metabolizing EA into Uro-A by intestinal flora relates to three kinds of enzymes: esterase, decarboxylase and catechol-dehydroxylase, and the core is catechol-dehydroxylase. The enzyme sequences possibly having these catalysis functions, including esterase (GenBank: VWA42738.1, as shown by SEQ ID NO.2) capable of hydrolyzing aromatic esters such as phenyl acetate to hydrolyze ester bonds into carboxyl and hydroxyl, gallic acid decarboxylase (UniProtKB: F9US27, as shown by SEQ ID NO.3) capable of respectively catalyzing gallic acid and protocatechuic acid to remove carboxyl from benzene rings to produce pyrogallol and catechol, dopamine dehydroxylase (GenBank: RDC23575.1, as shown by SEQ ID NO.4), hydrocaffeic acid dehydroxylase (GenBank: RDC18391.1, as shown by SEQ ID NO.5), catechin dehydroxylase (GenBank: RDC23615.1, as shown by SEQ ID NO.6), dopamine dehydroxylase (GenBank: RDB62136.1, as shown by SEQ ID NO.7), lignan dehydroxylase (GenBank: RDB65137.1, as shown by SEQ ID NO.8), etc. are obtained from NCBI. They have catechol-dehydroxylase enzymatic activity and may remove phenolic hydroxyl from different sites of benzene rings. The sequences of these enzymes were respectively subjected to BLAST alignment in a local genome database, and a total of 168 strains with a similarity of at least 70% and a coverage of at least 30% were screened out. Then, these strains were taken and respectively subjected to in vitro fermentation. After treatment on a fermentation broth, the detection was performed through a Waters liquid chromatograph (HPLC) and a QE high-resolution liquid chromatograph mass spectrometer (LCMS) to screen out strains capable of converting ETs to produce Uro-A. One strain was obtained in this screening process (a specific screening flow process is shown in FIG. 1).

### 2. Identification

A genome of the strain obtained through screening was extracted, 16S rDNA of the strain was subjected to amplification and sequencing (a nucleotide sequence of the 16S rDNA obtained through amplification was as shown by SEQ ID NO.1), the obtained sequence was subjected to nucleotide sequence alignment in NCBI-Blast, and the result showed that the strain is L. plantarum, and is named as L. plantarum CCFM1290.

Primers used for 16S rDNA amplification were as follows:
27F (forward): 5′-AGAGTTTGATCCTGGCCTCA-3′ (SEQ ID NO.9); and
1492R (reverse): 5′-GGTTACCTTGTTACGACTT-3′ (SEQ ID NO.10).
A 16S rDNA amplification procedure was as follows:
94° C. for 5 min; repeating for totally 30 cycles (94° C. for 30 s; 55° C. for 30 s; 72° C. for 2 min); 72° C. for 10 min; and 12° C. for 2 min.

### 3. Culture and Observation

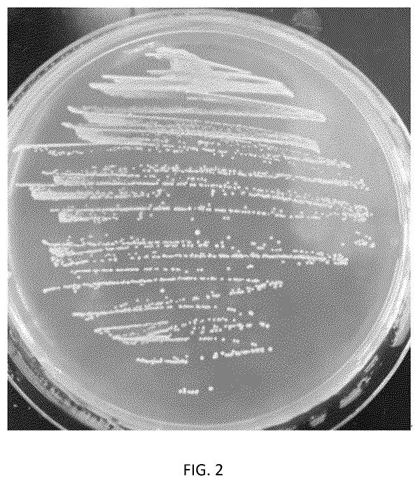

A single colony of the L. plantarum CCFM1290 was picked and inoculated into a BHI solid medium to be cultured for 48 h at 37° C., and the colony features of the strain on the BHI medium were observed (specific features are shown in FIG. 2).

Through observation, it can be seen that the bacterial colony of the L. plantarum on a BHI solid medium is raised, white, smooth, and round and has a diameter of about 3 mm.

### 4. Preservation

A single colony of the L. plantarum CCFM1290 was inoculated into a BHI liquid medium to be cultured for 36 h at 37° C., 1.0 mL of bacteria solution was taken and transferred into culture preservation tubes to obtain 5 parallel samples. 6000 rpm centrifugation was performed for 3 min. Supernatants were abandoned on super clean bench, 1.0 ml of 30% (m/m) sterile glycerin saline solution was added, and after the sufficient and uniform mixing by a vortex oscillator, the materials were preserved at −80° C.

### Example 2: Urolithin a Preparation Through Pomegranate Juice Fermentation by L. plantarum CCFM1290

1. Urolithin a Preparation by L. plantarum CCFM1290
(1) Activation of L. plantarum CCFM1290

A bacteria solution of L. plantarum CCFM1290 was dipped by an inoculating loop from the culture preservation tube to be streaked on a BHI solid medium, and the L. plantarum CCFM1290 was cultured for 24 h to 48 h in a constant temperature and humidity incubator under a 37° C. aerobic condition. A single colony was picked and inoculated into a BHI liquid medium to be cultured for 24 h under a 37° C. aerobic condition. A uniformly shaken liquid medium was taken and inoculated into a new BHI liquid medium at an inoculation amount of 2% (v/v) to be cultured. This operation was repeated for 3 times for activation culture, and an activated bacteria solution was obtained.

### (2) Preparation of Pomegranate Juice Fermentation Broth

A certain amount of commercially available NFC pomegranate juice was taken, 10 g/L of tryptone, 5 g/L of yeast powder, 2 g/L of K₂HPO₄ and 2 g/L of ammonium citrate dibasic were added, the pH value was adjusted to 7.0±0.2, sufficient oscillation was performed in a vortex oscillator, after 85° C. sterilization for 20 min, the materials were immediately put into a 4° C. environment for shock cooling so that thalli burst, and the materials were stored in a 4° C. environment for use in a week. Note: high-temperature sterilization (115° C., 20 min) may easily cause a serious Maillard reaction, so it was not included in the scope of the sterilization methods.

(3) Fermentation of L. plantarum CCFM1290

The activated bacteria solution obtained in step (1) was inoculated at an inoculation amount of 2% (v/v) into the pomegranate juice fermentation broth obtained in step (2), 200 rpm oscillation culture was performed in a 37° C. environment for 72 h, and the fermentation broth was respectively taken at 0 h, 18 h, 27 h, 48 h, and 72 h.

### 4. Uro-A Extraction

1 mL of fermentation broth was taken and respectively and continuously extracted for 3 times by 1 ml of diethyl ether and ethyl acetate. An extraction solution was treated to a nearly dry state by a refrigerated vacuum centrifugal concentrator, 1 ml of chromatographic grade methyl alcohol was added, 30 min ultrasonic solubilization was performed, and after being filtered through a 0.22 μm organic system filter membrane, the materials were transferred into a sample bottle, and the urolithin A (Uro-A) content was detected. The result was shown in Table 1.

TABLE 1
yield of Uro-A at different fermentation times

| | | Parallel | | | | | | | |
|---|---|---|---|---|---|---|---|---|---|
| Fermentation time | | 1 | | 2 | | 3 | | 4 | |
| 0 | h | 0.00 | μM | 0.00 | μM | 0.00 | μM | 0.00 | μM |
| 18 | h | 5.17 | μM | 6.40 | μM | 4.60 | μM | 5.87 | μM |
| 27 | h | 9.03 | μM | 11.22 | μM | 8.55 | μM | 10.25 | μM |
| 48 | h | 24.89 | μM | 26.51 | μM | 21.82 | μM | 25.59 | μM |
| 72 | h | 24.72 | μM | 26.03 | μM | 22.61 | μM | 24.89 | μM |

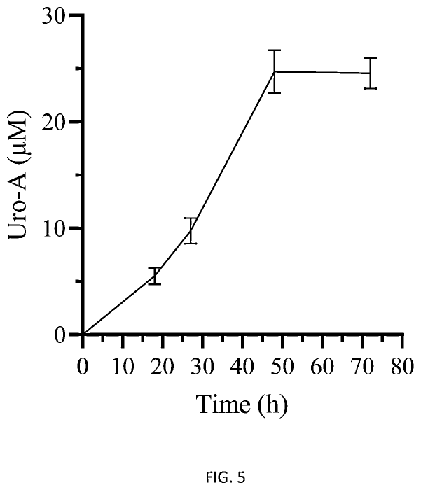

The result showed that the L. plantarum CCFM1290 may convert ETs (punicalagin) into Uro-A in pomegranate juice, and with the thallus growth, the content of Uro-A in the fermentation broth gradually increases. After the fermentation for 48 h, the Uro-A yield in the fermentation broth is basically kept in a stable state (FIG. 5).

2. Preparation of L. plantarum CCFM1290 Postbiotic

### (1) Postbiotic Preparation

A bacteria solution activated by the L. plantarum CCFM1290 was taken and inoculated at an inoculation amount of 2% (v/v) into the pomegranate juice fermentation broth, 200 rpm oscillation culture was performed in a 37° C. environment for 48 h, and the pomegranate juice fermentation broth at 48 h was respectively taken to be filtered, filter liquor was sterilized, and is subjected to 3000 rpm centrifugation for 15 min, supernatants were collected, and the postbiotic was obtained.

### Example 3: Preparation of Compound Preparation

Specific steps were as follows:

(1) Preparation of Bacteria Powder of L. plantarum CCFM1290

A bacteria solution of L. plantarum CCFM1290 was dipped to be streaked, and 37° C. aerobic inverted culture was performed for 18 h to 24 h. A single colony was picked to an MRS liquid medium, and after 200 rpm oscillation culture at 37° C. for 20 h, the materials were sufficiently and uniformly mixed. Then, the bacteria solution was taken and inoculated at an inoculation amount of 2% (v/v) to a new MRS liquid medium to be cultured in the same environment. This step was repeated for 3 times to 5 times, and a seed solution was finally prepared.

The prepared seed solution was inoculated at an inoculation amount of 2% to 4% (v/v) to an MRS liquid medium to be cultured for 18 h to 36 h. Centrifugation was performed, and bacterial sludge was collected, was sufficiently rinsed by a phosphate buffer solution for 3 to 5 times, was resuspended to 10¹⁰ CFU/mL by a freeze-drying protective additive, and was finally freeze-dried to obtain bacteria powder of L. plantarum CCFM1290.

In the above process, the freeze-drying protective additive included ingredients of 10% of skim milk powder, 3% of glycerinum, 10% of maltodextrin, and 15% of trehalose. These raw materials of the freeze-drying protective additive were mixed with drinking water and sufficiently dissolved, and sterilization was performed at 115° C. for 20 min.

(2) The bacteria powder of L. plantarum CCFM1290 in step (1), the pomegranate freeze-dried powder, and the freeze-dried mulberry powder were mixed according to a mass ratio of 5:1.5:0.5. Through detection, the viable count of L. plantarum CCFM1290 in a synbiotic preparation was (1.5-1.7)×10¹⁰ CFU/g.

### Example 4: Effects of Fermented Products and Compound Preparations on Caenorhabditis elegans

Specific steps were as follows:

1. Culture of E. coli OP50

A bacteria solution of E. coli OP50 was dipped by an inoculating loop from a culture preservation tube and was streaked on an LB solid medium, and the E. coli was cultured for 18 h in a constant temperature and humidity incubator under a 37° C. aerobic condition. A single colony was picked and inoculated into an LB liquid medium for 37° C. aerobic culture for 18 h in a constant temperature and humidity incubator. After uniform mixing, the materials were inoculated at an inoculating amount of 1.5% (v/v) into a new LB liquid medium to be cultured, and the operation was repeated for 3 times to obtain an activated bacteria solution.

2. Resuscitation, Passage, and Synchronization of Caenorhabditis elegans

One tube of dauer “dauer-stage” larval-phase Caenorhabditis elegans was taken out from a −80° C. refrigerator, fast thawed in a 37° C. water bath kettle, and then coated onto an NGM culture medium inoculated with E. coli OP50 to be cultured in a 20° C. constant temperature and humidity incubator. During the passage, 1 cm² of the NGM culture medium containing Caenorhabditis elegans was cut by a scalpel burnt by an alcohol lamp and pasted to a new NGM culture medium inoculated with the OP50.

The Caenorhabditis elegans on the NGM culture medium was collected by an M9 buffer solution, and excessive E. coli OP50 was washed away. After 10 min still standing, Caenorhabditis elegans bodies and eggs were precipitated to the bottom. Supernatants were abandoned, and the M9 buffer solution containing Caenorhabditis elegans, 5% NaClO, and 1 M NaOH were mixed according to a volume ratio of 2:1:2. Oscillation was continuously performed for 5 min on a vortex oscillator. After the Caenorhabditis elegans bodies were completely lysed, 6000 rpm centrifugalization was immediately performed for 2 min. Supernatants were abandoned, the M9 buffer solution was added for rinsing, and cleaning was repeated for 3 times to 5 times to sufficiently remove remained lysate. Finally, the Caenorhabditis elegans eggs were placed into 5 ml of an M9 buffer solution and subjected to 20° C. shake cultivation, and the Caenorhabditis elegans eggs basically completed incubation within 18 h to 24 h to obtain synchronous Caenorhabditis elegans.

3. Influence of Fermentation Product Obtained Through Pomegranate Juice Fermentation by L. plantarum CCFM1290 on Caenorhabditis elegans
(1) Fermentation Broth (Fermentation Product) Obtained Through Pomegranate Juice Fermentation by L. plantarum CCFM1290:

According to a method of step 1 in Example 2, the pomegranate fermentation broth fermented for 48 h obtained in step (3) was taken.

### (2) Unfermented Extracts of Pomegranate Juice:

A certain amount of commercially available NFC pomegranate juice was taken, 10 g/L of tryptone, 5 g/L of yeast powder, 2 g/L of K₂HPO₄ and 2 g/L of ammonium citrate dibasic were added, the pH value was adjusted to 7.0±0.2, sufficient oscillation was performed in a vortex oscillator, after 85° C. sterilization for 20 min, the materials were immediately put into a 4° C. environment for shock cooling so that thalli burst, and the materials were stored in a 4° C. environment for use in a week.

(3) The pomegranate juice fermentation broth/unfermented pomegranate juice was sufficiently extracted for 3 times by the same amount of diethyl ether and ethyl acetate and then subjected to refrigerated centrifugation concentration to a dry state, then, DMSO accounting for 1/100 of the volume of the original fermentation solution was added, finally, the materials were diluted to the original volume (i.e., a final concentration of DMSO was 1%) by an OP50 bacteria solution, and fermented extracts of pomegranate juice were obtained (Experiment group 1).

The unfermented extracts of pomegranate juice were a control group 1.

(4) 80 μL of the fermented extracts of pomegranate juice and the unfermented extracts of pomegranate juice obtained in step (3) were respectively taken and uniformly spread on a surface of an NGM culture medium to obtain intervention NGM culture medium respectively.

(5) The synchronous Caenorhabditis elegans obtained in step 2 was taken and cultured for 48 h in a normal NGM culture medium to an L4 phase. Then, the Caenorhabditis elegans was transferred to the intervention NGM culture medium obtained in step (4) to realize the intervention on the Caenorhabditis elegans, and after an average period of 2 days, the Caenorhabditis elegans was transferred to a new intervention type NGM culture medium. In the whole process from the L4 phase of the Caenorhabditis elegans to death, the Caenorhabditis elegans was exposed to compounds in the intervention type NGM culture medium.

Under the conditions that the temperature was 20° C., the culture medium was changed once every 2 days, and each culture medium for intervention additionally included 150 μM of 5-fluoro-2′-deoxyuridine (inhibiting the egg laying of Caenorhabditis elegans to prevent the influence of Caenorhabditis elegans filial generation on experiments), the intervention was continuously performed for 8 days.

### 4. Treatment of Compound Preparations

(1) The compound preparations prepared in Example 3 were Experiment group 2.

(2) E. coli OP50 bacteria powder (bacteria concentration was higher than or equal to 2.1×10¹⁰ CFU/g), pomegranate freeze-dried powder, and mulberry freeze-dried powder were mixed according to a mass ratio of 5:1.5:0.5 (after mixing, the OP50 viable count was higher than or equal to 1.5×10¹⁰ CFU/g), and the materials were used as Control group 2.

(3) Powder of Control group 2 and Experiment group 2 was respectively dissolved in water and then respectively coated to an NGM plate to intervene the growth of Caenorhabditis elegans:

The Caenorhabditis elegans, after synchronization, was cultured by a normal NGM culture medium for 48 h (L4 phase), and then, the Caenorhabditis elegans was transferred to the NGM culture medium added with the powder and obtained in the step for intervention. Additionally, in order to keep the concentration of extracts in the NGM culture medium stable, the Caenorhabditis elegans was transferred to the new NGM culture medium added with the powder once every two days.

Under the conditions that the temperature was 20° C., the culture medium was changed once every 2 days, and each culture medium for intervention additionally included 150 μM of 5-fluoro-2′-deoxyuridine (inhibiting the egg laying of Caenorhabditis elegans to prevent the influence of Caenorhabditis elegans filial generation on experiments), the intervention was continuously performed for 8 days.

### Example 5: Influence of Fermentation Products and Compound Preparations on Lifespan and Motion Ability of Caenorhabditis elegans

1. Detection on Lifespan of Caenorhabditis elegans

A specific embodiment was as Example 4. Experiment groups included Control group 1 (extracts of pomegranate juice), Control group 2 (E. coli OP50 bacteria powder+corresponding diet), Experiment group 1 (extracts of fermentation product) and Experiment group 2 (compound preparations).

In an intervention experiment process, the survival condition of the Caenorhabditis elegans was observed every day until the last Caenorhabditis elegans died.

During the lifespan test, the Caenorhabditis elegans was transferred once every 2 days so as to avoid bacteria contamination and keep the concentration of fermentation broth Uro in the plate.

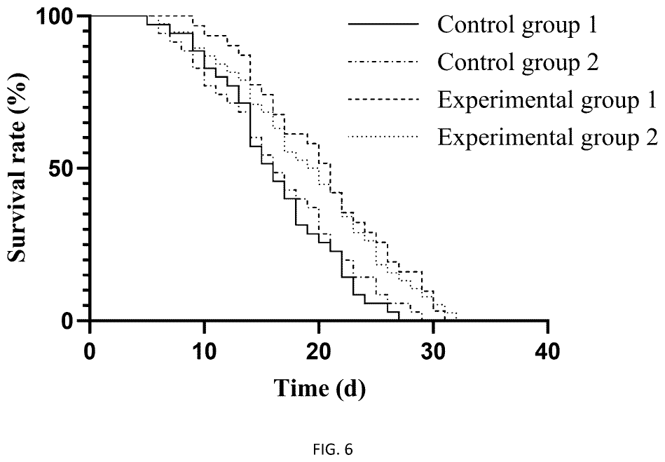

The result showed that (FIG. 6):

(1) Compared with Control group 1, Experiment group 1 (the extracts of fermentation product) achieves lifespan prolonging by 32.05% from the beginning of the Caenorhabditis elegans intervention from the L4 phase till the death of the Caenorhabditis elegans.

(2) Compared with Control group 2, Experiment group 2 (compound preparations) achieves lifespan prolonging by 21.88% from the beginning of the Caenorhabditis elegans intervention from the L4 phase till the death of the Caenorhabditis elegans.

2. Detection on Muscle State of Caenorhabditis elegans

Dexamethasone is a medicine that induces muscle damage in Caenorhabditis elegans. In the disclosure, the parietal muscle of the Caenorhabditis elegans was represented through the motion ability (swimming frequency) change of the Caenorhabditis elegans.

(1) A specific embodiment was as Example 4, and the differences were as follows:

(5) in step 3 was adjusted into the following procedure: the synchronous Caenorhabditis elegans obtained in step 2 was taken to be cultured in a normal NGM culture medium for 48 h to the L4 phase, and then, the Caenorhabditis elegans was transferred to the intervention type NGM culture medium obtained in step (4) to achieve intervention on the Caenorhabditis elegans. At the same time, 10 μM of dexamethasone was added into the culture medium, the Caenorhabditis elegans was transferred to a new intervention type NGM culture medium containing 10 μM of dexamethasone once every 2 days and cultured at 20° C. for 36 h.

(3) in step 4 was adjusted to the following procedure: after the Caenorhabditis elegans after synchronization was cultured for 48 h (L4 phase) by using a normal NGM culture medium, the Caenorhabditis elegans was transferred to the NGM culture medium added with the powder and obtained in the step for intervention. At the same time, 10 μM of dexamethasone was added into the culture medium. Additionally, in order to keep the concentration of the extracts in the NGM culture medium stable, the Caenorhabditis elegans was transferred to a new NGM culture medium containing the powder and 10 μM of dexamethasone once every two days and cultured at 20° C. for 36 h.

The blank group was as follows: the synchronous Caenorhabditis elegans obtained in step 2 was cultured in a normal NGM culture medium for 48 h to L4 phase, then, the Caenorhabditis elegans was transferred to a normal NGM culture medium to be cultured and cultured for 36 h at 20° C.

(2) After the culture was completed, the Caenorhabditis elegans in each group was transferred into NGM plates added with an M9 buffer solution (the pH value was 7.4). After the Caenorhabditis elegans adapted to the environment for 5 min, the swimming frequency of the Caenorhabditis elegans was recorded (the swinging motion of the Caenorhabditis elegans body from the left to the right and to the left was recorded as once swimming).

The result showed that:

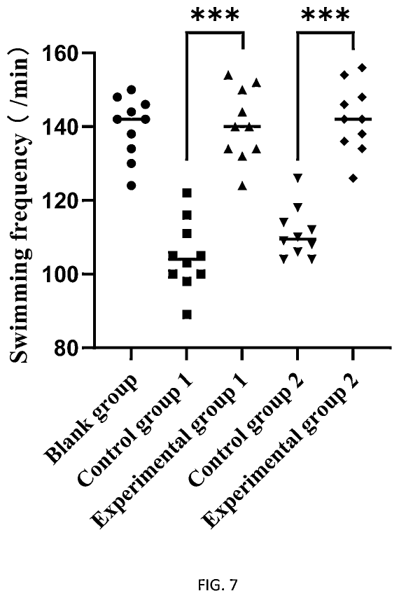

(1) Compared with Control group 1, the fermented extracts of product (Experiment group 1) improved the swimming frequency of the Caenorhabditis elegans by 33.84% (FIG. 7).

(2) Compared with Control group 2, compound preparations prepared in Example 3 (Experiment group 2) improved the swimming frequency of the Caenorhabditis elegans by 27.99% (FIG. 7).

It showed that the muscle health condition may be effectively improved through the fermented extracts of the product and the compound preparations.

### Example 6: Effect of L. plantarum on Caenorhabditis elegans

It is known that senescence may cause in vivo lipid metabolism disorder of Caenorhabditis elegans, so that abnormal accumulation of lipid droplets may be caused. Additionally, the lipid droplets may be easily invaded by ROS to cause lipid peroxidation, so that the cell senescence may be aggravated.

Specific steps were as follows:

1. Culture of E. coli OP50

A bacteria solution of E. coli OP50 was dipped by an inoculating loop from a culture preservation tube and streaked on an LB solid medium, and the E. coli was cultured for 18 h in a constant temperature and humidity incubator under a 37° C. aerobic condition. A single colony was picked and inoculated into an LB liquid medium to be cultured for 18 h in a constant temperature and humidity incubator under a 37° C. aerobic condition. After uniform mixing, the materials were inoculated at an inoculating amount of 1.5% (v/v) into a new LB liquid medium to be cultured, and the operation was repeated for 3 times to obtain an activated bacteria solution.

2. Resuscitation, Passage, and Synchronization of Caenorhabditis elegans

One tube of dauer “dauer-stage” larval-phase Caenorhabditis elegans was taken out from a −80° C. refrigerator, fast thawed in a 37° C. water bath kettle, and then coated onto an NGM culture medium inoculated with E. coli OP50 to be cultured in a 20° C. constant temperature and humidity incubator. During the passage, 1 cm² of the NGM culture medium containing Caenorhabditis elegans was cut by a scalpel burnt by an alcohol lamp and pasted to a new NGM culture medium inoculated with the OP50.

The Caenorhabditis elegans on the NGM culture medium was collected by an M9 buffer solution, and excessive E. coli OP50 was washed away. After 10 min of standing, Caenorhabditis elegans bodies and eggs were precipitated to the bottom. Supernatants were abandoned, and the M9 buffer solution containing Caenorhabditis elegans, 5% NaClO, and 1 M NaOH were mixed according to a volume ratio of 2:1:2. Oscillation was continuously performed for 5 min on a vortex oscillator. After the Caenorhabditis elegans bodies were completely lysed, 6000 rpm centrifugalization was immediately performed for 2 min. Supernatants were abandoned, the M9 buffer solution was added for rinsing, cleaning was repeated for 3 times to 5 times to sufficiently remove remained lysate. Finally, the Caenorhabditis elegans eggs were placed into 5 mL of an M9 buffer solution and subjected to 20° C. shake cultivation, and the Caenorhabditis elegans eggs basically completed incubation within 18 h to 24 h to obtain synchronous Caenorhabditis elegans.

3. Effect of L. plantarum on Caenorhabditis elegans

The Caenorhabditis elegans after synchronization grew 48 h to the L4 phase on an NGM plate inoculated with the E. coli OP50, and was then transferred to NGM culture media respectively coated with 1.0×10⁸ CFU/mL of E. coli OP50 and 1.0×10⁸ CFU/mL of L. plantarum. Additionally, 150 μM of FUDR (5-fluorouracil 2-deoxyriboside) was added into the culture media to inhibit egg laying of the Caenorhabditis elegans so that the influence of Caenorhabditis elegans filial generation on experiments might be avoided. Additionally, the Caenorhabditis elegans was transferred to a new NGM culture medium inoculated with the E. coli/L. plantarum once every 2 days to avoid interference by miscellaneous bacteria and ensure the concentration of strains (E. coli and L. plantarum) in the NGM culture medium.

At an aging phase (when being cultured to the 8th day), the Caenorhabditis elegans on the plates were cleaned by an M9 buffer solution and collected. A fixation solution was added into the collected solution for fixation for 30 min. The fixation solution was abandoned, an immediately made Oil red O staining solution was added, dip dyeing was performed for 20 min, and then, washing was performed for 3 times with an M9 buffer solution to remove the staining solution. Finally, the Oil red O staining intensity of the Caenorhabditis elegans was observed in a bright field under a microscope, and photos were taken. Next, the staining intensity was quantized by ImageJ software, and the accumulation level of Caenorhabditis elegans lipid droplets was represented by the average optical density.

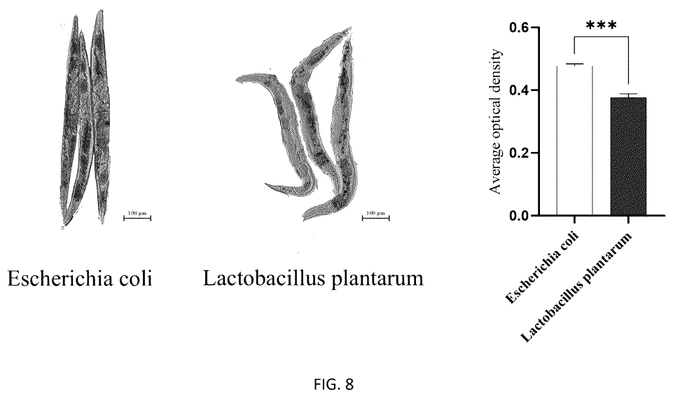

The result showed that compared with a condition of feeding the Caenorhabditis elegans with the E. coli OP50, a condition of feeding the Caenorhabditis elegans with the L. plantarum may obviously inhibit the lipid droplet accumulation in the Caenorhabditis elegans, and the lipid droplet content might be reduced by 21.21% (FIG. 8).

4. The Caenorhabditis elegans was provided with the E. coli OP50 and the FITC-labeled L. plantarum CCFM1290 at the same time, after 1 day, whether the Caenorhabditis elegans in took the L. plantarum CCFM1290 under the condition of existence of the E. coli OP50 was determined through determining the in vivo fluorescence of the Caenorhabditis elegans, and specific steps were as follows:

(1) FITC was prepared into a 1 mM solution by DMSO.

(2) The L. plantarum CCFM1290 grew to a state of OD600=0.6 in a 37° C. MRS culture solution and was then diluted to a state of OD600=0.3 by an MSR culture medium to obtain a bacterial culture medium.

(3) The prepared FITC solution was added into the bacterial culture medium at a proportion of 1% (v/v) and uniformly mixed (i.e., the final FITC spotting concentration was 100 μM), incubation was performed at 37° C. for 30 min, and then, free FITC was washed away by an M9 buffer solution.

(4) The FITC-labeled L. plantarum bacteria solution and the same amount of E. coli OP50 bacteria solution were mixed, and then the mixture was spread onto an NGM solid medium, after the solid medium was blown to a dry state, the synchronous Caenorhabditis elegans was transferred into the solid medium to be cultured for 1 day in a 20° C. dark environment, then, the Caenorhabditis elegans was flushed to an ep tube by an M9 buffer solution, the flushing was performed for 3 times with the M9 buffer solution to wash away excessive bacteria, and finally, whether the Caenorhabditis elegans in took the L. plantarum or not was detected through an inverted fluorescence microscope.

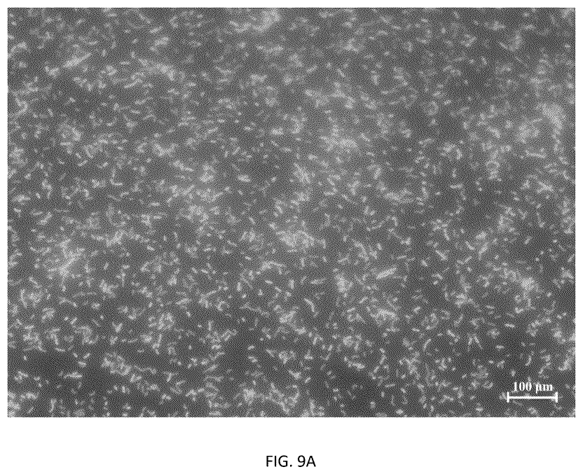

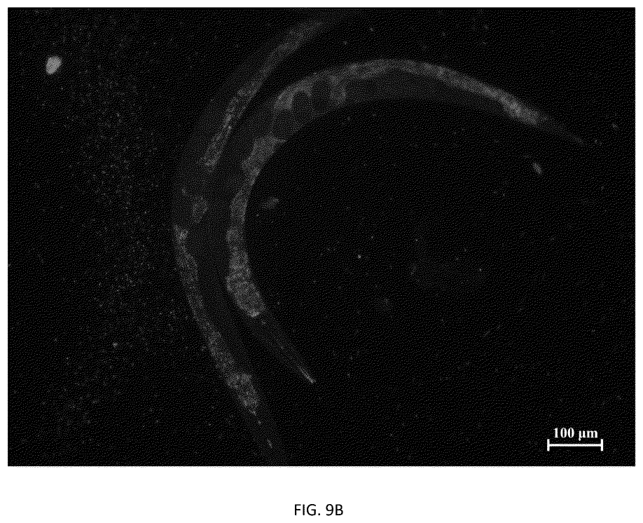

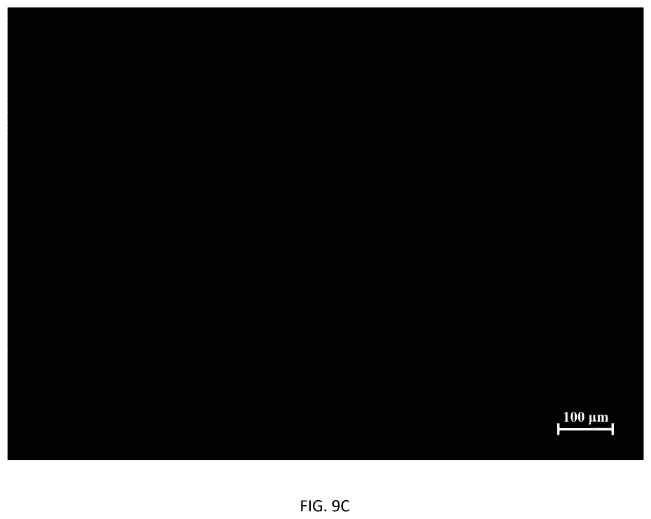

The result showed (FIG. 9) that through the fluorescence microscope detection on the FITC-labeled L. plantarum CCFM1290, it was proved that the L. plantarum CCFM1290 exists in the intestinal tracts of the Caenorhabditis elegans (b in FIG. 9), and it showed that under the condition that the E. coli OP50 exists, the Caenorhabditis elegans may also intake the L. plantarum CCFM1290, that is, when the L. plantarum CCFM1290 takes effects, the normal feeding of the Caenorhabditis elegans may not be influenced.

Although the exemplary examples of the disclosure have been provided above, they are not intended to limit the disclosure. Those skilled in the art will appreciate that various changes and modifications may be made without departing from the spirit and scope of the disclosure. Therefore, the protection scope of the disclosure shall be defined by the claims.

What is claimed is:

1. A composition, comprising the L. plantarum CCFM1290; wherein the L. plantarum CCFM1290 is preserved in Guangdong Microbial Culture Collection Center (GDMCC) with a preservation number of GDMCC No: 62801 and a preservation date of Sep. 15, 2022.

2. The composition according to claim 1, wherein the composition is a food, a medicine, a microbial preparation, a dietary supplement, or a health care product.

3. The composition according to claim 2, wherein the food is a health care food or the food is a dairy product or a bean product or a fruit and vegetable product produced by using a starter culture containing the L. plantarum CCFM1290; or the food is a beverage or snacks containing the L. plantarum CCFM1290.

4. The composition according to claim 2, wherein the microbial preparation comprises the L. plantarum CCFM1290, or comprises a fermentation broth containing the L. plantarum CCFM1290, or freeze-dried powder containing the L. plantarum CCFM1290, or inactivated thalli containing the L. plantarum CCFM1290, or a lysate containing the L. plantarum CCFM1290, or an extract containing the L. plantarum CCFM1290.

5. The composition according to claim 4, wherein the content of the L. plantarum CCFM1290 in the microbial agent is not lower than 10⁹ CFU/mL or 10¹⁰ CFU/g.

6. The composition according to claim 1, being a medicine capable of delaying senescence, wherein the medicine comprises the L. plantarum CCFM1290.

7. A method for preparing urolithin A, comprising: using a substance containing ellagitannins (ETs) or ellagic acid (EA) as a substrate, inoculating the composition of claim 4 in a culture and fermenting the culture to prepare a reaction solution, and then performing extraction from the reaction solution to obtain the urolithin A.

8. The method according to claim 7, wherein the substance containing the ETs comprises one or more of pomegranates, mulberry mulberries, raspberries, strawberries, walnuts, pistachio nuts, or corcassian walnuts.

9. The method according to claim 7, wherein the reaction conditions comprise a temperature of 30° C. to 37° C., a revolution velocity of 150 rpm to 200 rpm, and the time of 24 hours to 72 hours.

10. A method for fermenting pomegranate juice, comprising the following steps:
(1) activating L. plantarum CCFM1290:
dipping a bacteria solution of the L. plantarum CCFM1290 for streaking on a Brain Heart Infusion Broth (BHI) solid medium, and culturing the L. plantarum CCFM1290 for 24 hours to 48 hours in a constant temperature and humidity incubator under a 37° C. aerobic condition; and picking a single colony to be inoculated into a BHI liquid medium for 37° C. aerobic culture for 24 hours, inoculating the well shaken liquid medium into a new BHI liquid medium at an inoculation amount of 2% to be cultured, and repeating the operation for 3 times for activation culture to obtain an activated bacteria solution; wherein the L. plantarum CCFM1290 is preserved in Guangdong Microbial Culture Collection Center (GDMCC) with a preservation number of GDMCC No: 62801 and a preservation date of Sep. 15, 2022
(2) preparing pomegranate juice fermentation broth:
taking a certain amount of commercially available pomegranate juice, adding 10 g/L of tryptone, 5 g/L of yeast powder, 2 g/L of K₂HPO₄, and 2 g/L of ammonium citrate dibasic, adjusting the pH value to 7.0±0.2, performing sufficient oscillation in a vortex oscillator, after sterilization at 85° C. for 20 min, immediately placing the material into a 4° C. environment to be subjected to shock cooling so that thalli burst, storing the material in a 4° C. environment for use in a week, and obtaining the pomegranate juice fermentation broth;
(3) fermenting L. plantarum pomegranate juice:
inoculating the activated bacteria solution obtained in Step (1) into the pomegranate juice fermentation broth obtained in Step (2) at an inoculating amount of 2%, and performing an oscillation culture for 72 h at a revolution velocity of 200 rpm in a 37° C. environment.
